(19) United States
(12) Patent Application Publication
(10) Pub. No.: US 2017/0121754 A1
(43) Pub. Date: May 4, 2017

(54) Test Strip, Meter, and Method for Assaying Enzyme Activity

(71) Applicant: Abbott Diabetes Care Inc., Alameda, CA (US)

(72) Inventors: Benjamin J. Feldman, Berkeley, CA (US); Tianmei Ouyang, Fremont, CA (US)

(21) Appl. No.: 15/310,685

(22) PCT Filed: Jun. 4, 2015
(86) PCT No.: PCT/US15/34238

§ 371 Date: Nov. 11, 2016

Related U.S. Application Data
(60) Provisional application No. 62/015,117, filed on Jun. 20, 2014.

(51) Int. Cl. C12Q 1/32; C12Q 1/00; G01N 27/327

(52) U.S. Cl. CPC C12Q 1/32; G01N 27/3272; G01N 27/3273; C12Q 1/004; G01N 2333/904

(57) ABSTRACT

Embodiments of the present disclosure relate to an electrochemical test strip comprising a reagent system for assaying activity of an enzyme that may be present is a sample. The present disclosure also provides a meter with a port for receiving the test strip and electronics for determining activity of the enzyme, if present, in the sample. Method for using the test strip for determining activity of the enzyme is also described.

### CROSS-REFERENCE TO RELATED APPLICATION

This application claims priority based on U.S. Provisional Application No. 62/015,117, filed Jun. 20, 2014, the disclosures of which is incorporated by reference herein in its entirety.

### INTRODUCTION

A simple and rapid test for activity of an enzyme, such as, an enzyme present in a body fluid of a patient would greatly simplify screening, treatment, diagnosis, and/or prognosis of patients.

Tests performed at the point of care (POC) have become common and very useful tools not only in health care facilities including hospitals, doctors′, and nurses' offices but also in other sites including, remote field sites or workplaces.

As such, there is interest in the art in obtaining test strips that can provide a rapid assay of activity of an enzyme of interest.

### SUMMARY

Embodiments of the present disclosure relate to an electrochemical test strip comprising a reagent system for assaying activity of an enzyme that may be present is a sample. The present disclosure also provides a meter, with a port for receiving the test strip, for determining activity of the enzyme, if present, in the sample. Method for using the test strip for determining activity of the enzyme is also described.

In certain embodiments, an electrochemical test strip for assaying activity of an enzyme is provided. The electrochemical test strip may include a first layer, a second layer and a spacer layer interposed between the first and second layers; a sample chamber defined by the first, second, and spacer layers, and positioned between the first and second layers. The sample chamber may include a substrate for the enzyme and a redox mediator disposed in the sample chamber; and a first electrode and a second electrode.

In certain embodiments, the substrate may be glucose-6-phosphate and the enzyme may be glucose-6-phosphate dehydrogenase. In certain embodiments, the sample chamber comprises an erythrocyte lysing agent. In certain embodiments, the substrate may be glucose-6-phosphate, lactate, hemoglobin, pyruvate, creatinine, aspartate, alanine, urea, or cholesterol.

In certain embodiments, the first layer or the second layer may be opaque or transparent. The first and second electrodes may be disposed on the first layer or the second layer. In certain embodiments, one of the electrodes is disposed on the first layer and the other electrode is disposed on the second layer. In certain embodiments, a third electrode may be included in the sample chamber. The third electrode may be disposed on the first or the second substrate. In certain cases, the first, second, and third electrodes may be working, counter, and reference electrodes, respectively.

In certain embodiments, the first electrode may be a working electrode and the second electrode may be a counter electrode, wherein the electrodes independently comprise a material selected from the group consisting of: gold, carbon, platinum, tin oxide, ruthenium, palladium, silver, silver chloride, silver bromide, and combinations thereof. In certain cases, the counter electrode may include Ag and AgCl.

In certain cases, the first electrode may be a working electrode and may be disposed on the first layer and the second electrode may be a counter electrode and may be disposed on the second layer. In other cases, the first electrode may be a working electrode and the second electrode may be a counter electrode and both electrodes may be disposed on the first layer or on the second layer. The substrate and redox mediator may be disposed on the first electrode or the second electrode.

In certain cases, the redox mediator may be phenazine methosulfate or a mediator that includes a transition metal complex. The transition metal complex ma include a transition metal selected from the group consisting of osmium, ruthenium, iron and cobalt. In certain cases, the transition metal may be osmium.

In certain cases, test strip he sample chamber may include a cofactor for the enzyme being assayed.

The volume of the sample chamber may be about 15 μL or less, e.g., 0.1 μL. The sample chamber may have a height of about 250 μm to about 15 μm. e.g., 100 μm to about 40 μm.

Also disclosed herein is a kit that may include a test strip as described here and a control solution that includes the enzyme that is assayed by the test strip. The control solution may include the enzyme at a concentration close to a normal physiological concentration in a human.

A system for assaying activity of an enzyme is also provided. The system may include the test strip as described herein and a meter compatible with the test strip. The meter may include electronics for measuring a signal generated by action of the enzyme on the substrate. The signal may be current, resistance, impedance, voltage, or capacitance. The meter may include a display for displaying the results of the assaying the activity of the enzyme.

A method for assaying activity of an enzyme is also disclosed. The method may include contacting a sample suspected of containing the enzyme with a first test strip, where the first test strip is the test strip as disclosed herein; and measuring a signal generated by action of the enzyme on the substrate, where the presence of the signal indicates that the enzyme is present in the sample; and where magnitude of the signal is proportional to the concentration of the enzyme present in the sample.

In exemplary embodiments, prior to or after the contacting the sample, the method may include contacting a second test strip with a control solution, wherein the second test strip is the test strip as disclosed herein, and where the control solution includes a known amount of the enzyme.

The method may include determining concentration of the enzyme by coulometry or amperometry.

The sample may be a body fluid of a subject, e.g., whole blood. The whole blood sample may be obtained from the finger of the subject. In certain cases, the whole blood sample may be obtained from a region of the subject having a lower nerve end density as compared to a fingertip, such as, abdomen or forearm.

### BRIEF DESCRIPTION OF THE DRAWINGS

A detailed description of various embodiments of the present disclosure is provided herein with reference to the accompanying drawings, which are briefly described below. The drawings are illustrative and are not necessarily drawn to scale. The drawings illustrate various embodiments of the present disclosure and may illustrate one or more embodiment(s) or example(s) of the present disclosure in whole or in part. A reference numeral, letter, and/or symbol used in one drawing to refer to a particular element may be used in another drawing to refer to a like element.

FIG. 1 depicts an embodiment of a test strip as described herein.

FIG. 2 depicts an embodiment of a test strip as described herein.

FIG. 3 illustrates a test strip showing a first layer and a second layer with a spacer layer.

FIG. 4 depicts individual elements of a test strip as described herein.

FIGS. 5A-C depict an embodiment of a test strip as described herein.

FIG. 6 provides a schematic of a test strip described herein.

FIG. 7 illustrates a modified FreeStyle strip for enzyme activity assay.

FIG. 8 provides a schematic of the transfer of electrons from glucose-6-phosphate (G6P) to the working electrode.

FIG. 9 shows current generated by glucose-6-phosphate dehydrogenase (G6PDH) activity.

FIG. 10 depicts a plot of the steady-state current value vs. G6PDH activity.

FIG. 11 provides a schematic of the transfer of electrons from glucose-6-phosphate (G6P) to the working electrode.

FIG. 12 shows current generated by glucose-6-phosphate dehydrogenase (G6PDH) activity.

FIG. 13 shows current generated by glucose-6-phosphate dehydrogenase (G6PDH) activity measured in whole blood and lysed blood.

FIG. 14 shows current generated by glucose-6-phosphate dehydrogenase (G6PDH) activity in blood lysed within the test strips disclosed herein.

### DETAILED DESCRIPTION OF THE INVENTION

Before the embodiments of the present disclosure are described, it is to be understood that this invention is not limited to particular embodiments described, as such may, of course, vary. It is also to be understood that the terminology used herein is for the purpose of describing particular embodiments only, and is not intended to be limiting, since the scope of the embodiments of the invention will be embodied by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

In the description of the invention herein, it will be understood that a word appearing in the singular encompasses its plural counterpart, and a word appearing in the plural encompasses its singular counterpart, unless implicitly or explicitly understood or stated otherwise. Merely by way of example, reference to “a” or “the” “sample” encompasses a single sample, as well as a two or more different samples, reference to “a” or “the” “test strip” encompasses a single assay, as well as two or more test strips, and the like, unless implicitly or explicitly understood or stated otherwise. Further, it will be understood that for any given component described herein, any of the possible candidates or alternatives listed for that component, may generally be used individually or in combination with one another, unless implicitly or explicitly understood or stated otherwise. Additionally, it will be understood that any list of such candidates or alternatives, is merely illustrative, not limiting, unless implicitly or explicitly understood or stated otherwise.

Various terms are described below to facilitate an understanding of the invention. It will be understood that a corresponding description of these various terms applies to corresponding linguistic or grammatical variations or forms of these various terms. It will also be understood that the invention is not limited to the terminology used herein, or the descriptions thereof, for the description of particular embodiments. Merely by way of example, the invention is not limited to particular enzymes, bodily or tissue fluids, blood or capillary blood, or test strip design or usages, unless implicitly or explicitly understood or stated otherwise, as such may vary.

The publications discussed herein are provided solely for their disclosure prior to the filing date of the application. Nothing herein is to be construed as an admission that the embodiments of the invention are not entitled to antedate such publication by virtue of prior invention. Further, the dates of publication provided may be different from the actual publication dates which may need to be independently confirmed.

Generally, embodiments of the present disclosure relate to an electrochemical test strip comprising a reagent system for assaying activity of an enzyme that may be present in a sample. The present disclosure also provides a meter for measuring a signal from a test strip of the present disclosure and for determining activity of the enzyme, if present, in the sample. Method for using the test strip for determining activity of the enzyme is also described. Methods of making the disclosed test strips are also provided.

### Electrochemical Test Strips

An electrochemical test strip that can be used to assay activity of an enzyme is described. The test strip may be used to assay activity of an enzyme and determine presence of the enzyme in a sample and/or measure the amount of the enzyme.

The electrochemical test strip may include a first layer, a second layer and a spacer layer interposed between the first and second layers; a sample chamber defined by the first, second, and spacer layers, and positioned between the first and second layers. The sample chamber may include a reagent system for assaying activity of an enzyme of interest.

In certain embodiments, the reagent system may include a substrate for the enzyme and a redox mediator, and optionally, a cofactor for the enzyme. The reagent system may be disposed in the sample chamber. In certain embodiments, the reagent system may be disposed on or near an electrode present in the sample chamber. For example, the sample chamber may include a working electrode and a counter electrode, and the reagent system may be disposed on or in vicinity of the working electrode.

The components of the reagent system are specific to the enzyme that is to be assayed using the test strip. The test strips disclosed herein are suitable for assaying a numerous different enzymes of interest. In certain cases, enzyme may be one of glucose-6-phosphate dehydrogenase (G6PDH or G6PD), lactate dehydrogenase (LDH), alanine aminotransferase (ALT), aspartate aminotransferase (AST), pyruvate kinase (PK), glucocerebrosidase, cholesterol oxidase, β-hydroxybutyrate dehydrogenase, alcohol dehydrogenase, formaldehyde dehydrogenase, malate dehydrogenase, 3-hydroxysteroid dehydrogenase or creatinine kinase (CK).

As noted above, the reagent system may include a substrate for the enzyme of interest. The substrate may be a naturally occurring substrate or a modified substrate, e.g., a substrate that is specific to the enzyme but includes modification to increase its binding to the cognate active site on the enzyme, increase the stability of the naturally occurring substrate, and the like. Exemplary substrates, any of which may be disposed in the sample chamber of the test strips disclosed herein include, glucose-6-phosphate (G6P), lactate, pyruvate, creatinine, aspartate, alanine, glucocerebroside, hemoglobin, urea, or cholesterol.

In certain embodiments, the enzyme of interest may optimally catalyze the conversion of the substrate into a reaction product in the presence of a cofactor, a coenzyme, an activator of the enzyme, or a combination thereof. In such cases, the reagent system may optionally include the cofactor, the coenzyme, the activator of the enzyme, or a combination thereof.

As is known in the field of electrochemical test strips, mediators facilitate the transfer of electrons generated from action of an enzyme on its substrate to the electrodes of the test strips. Any of a variety of mediators may be included in the test strips disclosed herein. In certain cases, the mediators may have low reactivity with oxygen. However, in certain cases, mediators that react with oxygen may be included. Exemplary mediators, any of which may be disposed on or in proximity to the working electrode in the sample chamber are disclosed below.

As noted above, the components of the reagent system included in the test strip depend on the enzyme to be detected. Exemplary, reagent system for G6PD include the following components: (i) G6P, a cofactor, e.g., nicotinamide adenine dinucleotide (NAD), and a mediator for oxidizing NADH, e.g., phenazine methosulfate; or (ii) G6P, a cofactor, e.g., nicotinamide adenine dinucleotide phosphate (NADP), and a mediator for oxidizing NADPH, e.g., phenazine methosulfate.

In certain cases, the reagent system may include an enzyme that may facilitate transfer of electrons, generated from oxidation of the substrate by the enzyme, from the cofactor to a mediator. For example, the reagent system may include diaphorase which may facilitate transfer of electrons from NADH or NADPH to a mediator, such as, an Osmium based mediator.

In exemplary embodiments, a reagent system for detecting activity of G6PD may include G6P, NAD or NADP, diaphorase, and an Osmium based mediator.

In certain cases, the sample chamber may additionally include a buffer, e.g., 4-(2-hydroxyethyl)-1-piperazineethanesulfonic acid (HEPES), tris(hydroxymethyl) aminomethane (TRIS), TRIS-hydrochloride (TRIS-HCl), phosphate buffered saline (PBS), piperazine-N,N′-bis(2-ethanesulfonic acid) (PIPES), and the like, or a combination thereof. In certain test strips of the present disclosure, the sample chamber may also include a surfactant, e.g., TWEEN-20, TRITON X-100, FS-30, or a combination thereof.

As noted above, the reagent system may be disposed on or near the working electrode. In certain cases, the working electrode and/or the counter electrode may have a buffer and/or surfactant disposed thereupon. The buffer disposed on the counter electrode may be the same or different from the buffer disposed on the working electrode.

In certain cases, the sample in which the enzyme activity is to be assayed may be a body fluid sample, such as, a blood sample, e.g., venous or arterial blood sample, plasma, serum, interstitial fluid, urine, tear, saliva, cerebrospinal fluid, semen, or milk. In exemplary embodiments, the body fluid sample may be blood.

In certain cases, the enzyme being assayed with the test strip may not be readily accessible to the reagent system disposed in the sample chamber. For example, the majority of the enzyme may be present inside a cell. The enzyme may be released from the cell or made accessible to the reagent system by lysing the cell. Cell lysis may be performed on the sample prior to contacting it with the test strip. In other embodiments, cell lysis may be carried out in the sample chamber of the test strip.

In certain embodiments, the sample chamber may include a cell lysis reagent, such as an erythrocyte lysis reagent. The erythrocyte lysis reagent may be saponin, ammonium chloride based lysis reagent, hemolysin, and the like. The cell lysis reagent may be disposed in the sample chamber on an electrode surface and/or the first or second layer. The amount of erythrocyte lysis reagent disposed in a sample chamber may be about 50 μg-1 μg, e.g., 45 μg, 30 μg, 20 μg, 10 μg, 5 μg, 3 μg, or 2 μg. In certain cases, the amount of erythrocyte lysis reagent disposed in the sample chamber may be determined based on the volume of the sample chamber. For example, for a 1 μL sample chamber, 30 μg-1 μg erythrocyte lysis reagent may be disposed in the sample chamber.

Other exemplary reagent systems for assaying activity of an enzyme include a reagent system for assaying activity of lactate dehydrogenase. This reagent system may include the following components: lactate, NAD and a mediator.

The test strips of the present disclosure may be used to measure amount of enzyme present in a sample by measuring the activity it catalyzes. In other words, the test strips measure the concentration of the enzyme in terms of its activity. As such, the concentration may not be an absolute concentration by rather a measure of amount of active enzyme. For example, the test strip may be used to distinguish the activity of an enzyme and a mutant thereof that has reduced activity, when the samples include the same amount of the enzyme and the mutant in terms of the level of the protein.

In certain cases, the test strips disclosed herein may be used to measure enzyme unit (U) of an enzyme of interest. One U may be defined as the amount of the enzyme that produces a certain amount of enzymatic activity, for example, the amount that catalyzes the conversion of 1 micro mole of substrate per minute under specified conditions.

The components of the reagent system are present in excess of the amount of enzyme that may be present in the sample. In exemplary test strips, the components of the reagent system are present in an amount that facilitate a catalytic reaction that is a “zero order” reaction because the rate of conversion of the substrate into a reaction product is independent of substrate concentration—the addition of more substrate does not serve to increase the rate.

In certain embodiments, the test strips in combination with a measuring device provide a detection and/or measurement of enzyme activity within 5 min of contacting the test strip with the sample. e.g., within 4 min, 3 min, 2 min, 60 sec, 45 sec, 30 sec, 15 sec, or less.

The construction of test strip may be similar to other electrochemical test strips known in the literature, such as, glucose test strips. Substrates and/or insulative layer or spacer layer of the test strips used in the methods disclosed herein may be made of a flexible polymer, such as a polyester (e.g., Mylar™ and polyethylene terephthalate (PET)), polyethylene, polycarbonate, polypropylene, nylon, polyvinyl chloride (PVC), polyurethanes, polyethers, polyamides, polyimides, or copolymers of these thermoplastics, such as PETG (glycol-modified polyethylene terephthalate).

In other embodiments, the sensors are made using a relatively rigid substrate to, for example, provide structural support against bending or breaking Examples of rigid materials that may be used as the substrate include glass, poorly conducting ceramics, such as aluminum oxide and silicon dioxide.

The electrodes may be made of any conductive material such as pure metals or alloys, or other conductive materials. Examples include aluminum, carbon (such as graphite), cobalt, copper, gallium, gold, indium, iridium, iron, lead, magnesium, mercury (as an amalgam), nickel, niobium, osmium, palladium, platinum, rhenium, rhodium, selenium, silicon (such as highly doped polycrystalline silicon), silver, tantalum, tin, titanium, tungsten, uranium, vanadium, zinc, zirconium, mixtures thereof, and alloys or metallic compounds of these elements. In certain embodiments, the conductive material includes carbon, gold, platinum, palladium, iridium, or alloys of these metals, since such noble metals and their alloys are unreactive in biological systems. In certain cases, the reference electrode or the reference/counter electrode may be a silver/silver chloride electrode.

Electrodes (and/or other features) may be applied or otherwise processed using any suitable technology, e.g., chemical vapor deposition (CVD), physical vapor deposition, sputtering, reactive sputtering, printing, coating, ablating (e.g., laser ablation), painting, dip coating, etching, and the like.

In certain embodiments, the thickness of spacer layer may be constant throughout, and may be at least about 0.01 mm (10 μm) and no greater than about 1 mm or about 0.5 mm. For example, the thickness may be between about 0.02 mm (20 μm) and about 0.25 mm (250 μm), e.g., 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, or 250 μm. In one certain embodiment, the thickness is about 0.05 mm (50 μm), and about 0.1 mm (100 μm) in another embodiment.

The sample chamber has a volume sufficient to receive a sample of biological fluid therein. In some embodiments, the sample chamber has a volume that is typically no more than about 20 μL. For example, no more than about 15 μL, 10 μL, 5 μL, 3 μL, 2 μL, 1 μL, 0.8 μL, or 0.5 μL, and also for example, no more than about 0.3 μL, 0.25 μL, or 0.1 μL.

The components of the reagent system may be deposited as an aqueous solution and subsequently dried, e.g., air dried or deposited in a dry form. The components of the reagent system can be screen-printed, slot coated, deposited using an ink jet, for example. The components of the reagent system can be deposited individually or in combinations of two or more components or as a single layer, for example, using a solution that includes all components of the reagent system.

A layer of mesh may overlay the electrode(s). This layer of mesh may protect the sensing layer from physical damage. The layer of mesh may also facilitate wetting the electrodes by reducing the surface tension of the sample, thereby allowing it to spread evenly over the electrodes. The mesh layer may also facilitate filling of the sample chamber by wicking the sample into the sample chamber. The mesh layer may be made of a polymer.

In certain embodiments, the sensor may not include a mesh layer that may filter out blood cells, such as, red blood cells. In certain embodiments, the sensor does not include a layer, such as a membrane, that may filter out the blood cells from a sample.

Test strips can be of any construction, size, or shape known to those skilled in the art; for example, FREESTYLE® and FREESTYLE LITE™ test strips, as well as PRECISION™ test strips sold by ABBOTT DIABETES CARE Inc., ACCU-CHEK Aviva test strips, ACCU-CHEK Aviva Plus test strips, CONTOUR® test strips, BREEZE® 2 test strips, OneTouch® test strips, OneTouch® Ultra® test strips may be modified to replace their sensing chemistry with the reagent system described herein. In addition to the embodiments specifically disclosed herein, the reagents and methods of the present disclosure can be configured to work with a wide variety of analyte test strips, e.g., those disclosed in U.S. Pat. No. 7,866,026; U.S. Patent Application Publication No. 2007/0095661; U.S. Patent Application Publication No. 2006/0091006; U.S. Pat. No. 7,918,975; U.S. Pat. No. 6,616,819; U.S. Pat. No. 6,143,164; U.S. Pat. No. 6,592,745; U.S. Pat. No. 6,071,391 and U.S. Pat. No. 6,893,545; U.S. Pat. No. 5,628,890; U.S. Pat. No. 6,764,581; and U.S. Pat. No. 7,311,812, for example, the disclosures of each of which are incorporated by reference herein in their entirety.

The terms “working electrode”, “counter electrode”, “reference electrode” and “counter/reference electrode” are used herein to refer to a portion or portions of a conductive trace which are configured to function as a working electrode, counter electrode, reference electrode or a counter/reference electrode respectively. In other words, a working electrode is that portion of a conductive trace which functions as a working electrode as described herein, e.g., that portion of a conductive trace which is exposed to an environment containing the enzyme to be assayed and not covered by an insulative layer (such as a spacer layer, a tape, or a cover). Similarly, a reference electrode is that portion of a conductive trace which function as a reference electrode as described herein, e.g., that portion of a conductive trace which is exposed to an environment containing the enzyme to be assayed and not covered by an insulative layer, and which, in some cases, includes a secondary conductive layer, e.g., a Ag/AgCl layer. A counter electrode is that portion of a conductive trace which is configured to function as a counter electrode as described herein, e.g., that portion of a conductive trace which is exposed to an environment containing the enzyme to be assayed to be measured and not covered by an insulative layer. In some embodiments, a portion of a conductive trace may function as either or both of a counter electrode and a reference electrode.

The dimensions of the test strip may vary. In certain embodiments, the overall length of test strips may be no less than about 10 mm and no greater than about 50 mm. For example, the length may be between about 30 and 45 mm; e.g., about 30 to 40 mm. It is understood, however that shorter and longer sensor strips could be made. In certain embodiments, the overall width of sensor strip may be no less than about 3 mm and no greater than about 15 mm. For example, the width may be between about 4 and 10 mm, about 5 to 8 mm, or about 5 to 6 mm. In one particular example, test strip has a length of about 32 mm and a width of about 6 mm. In another particular example, sensor strip has a length of about 40 mm and a width of about 5 mm. In yet another particular example, sensor strip has a length of about 34 mm and a width of about 5 mm.

It is noted that the terms, “sensor”, “sensor strip”, “biosensor”, “electrochemical test strip”, or “test strip”, are used interchangeably herein.

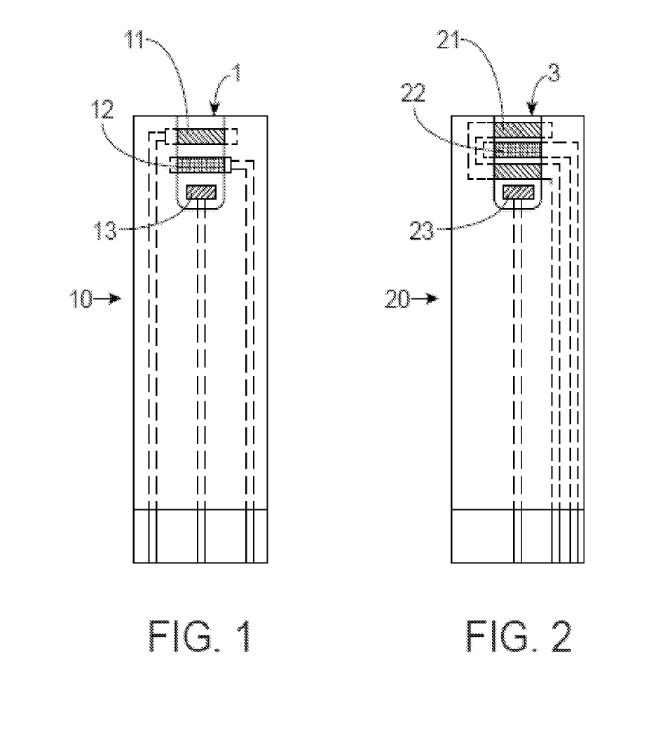

Exemplary test strips are depicted in FIGS. 1-6. The test strip may include a first, second and a third electrode as illustrated in FIGS. 1-3, and 6. For example, as shown in the sensor 10 of FIG. 1, the first electrode 11 may be closest to the sample application site 1, followed by the second electrode 12, and third electrode 13. The sensor in FIG. 1 is depicted as having a first substrate onto which the electrodes are disposed and further having an insulative layer, with a cut-out for the sample chamber, disposed on the electrodes, the cut-out exposes the electrodes in the sample chamber while covering other portions of the electrodes. Accordingly, within the sample chamber, the electrodes are disposed such that a sample applied at the tip of the sensor at application site 1, contacts the first electrode 11 first, then the second electrode 12, and then the third electrode 13. The conductive trace portions of the electrodes which connect the electrodes to a meter are covered by the insulative layer. These sensors may have an additional layer, such as a second substrate disposed over the insulative layer. The cut out in the insulative layer and the first and second substrates defining the sample chamber.

In other embodiments, the first electrode may be on a first substrate of the sensor while the second and/or the third electrode may be on a second substrate of the sensor, where the arrangement of the electrode with regard to the sample application site may be as described above.

FIG. 2 shows the configuration of the electrodes in test strip 20. The test strip includes a dual purpose counter/reference electrode 21 that wraps around a working electrode 22. A trigger electrode 23 is disposed downstream to indicate filling of the sample chamber by a sample. Numeral 3 indicates the sample application site.

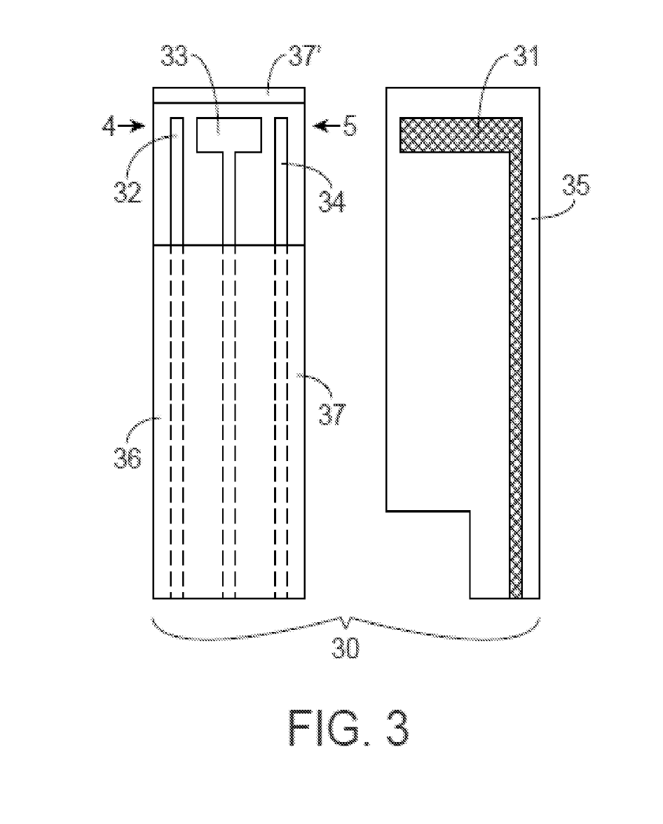

In other embodiments, the test strip may be as shown in FIG. 3. In FIG. 3, the test strip 30 includes a first electrode 31 on a first substrate 35; and a second electrode 32, third electrode 33, and fourth electrode 34 on a second substrate 36. In the assembled test strip, the first electrode is a facing orientation to electrodes 32, 33, and 34. In the sensor of FIG. 3, the sample may be filled from either side entrance 4 or 5. A spacer layer 37 and 37′ in combination with the two substrates 35 and 36 define the sample chamber.

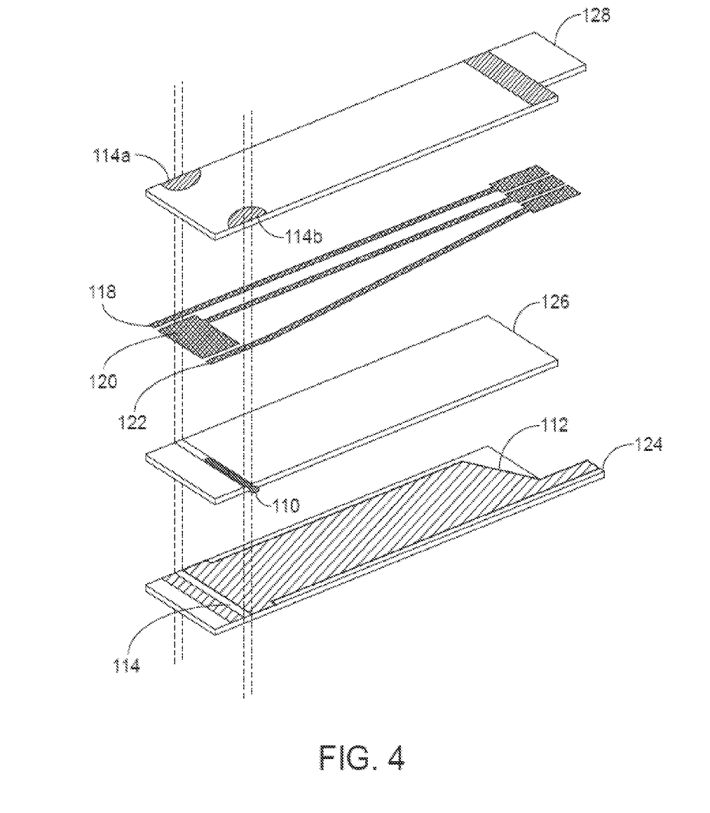

An exploded view of a test strip having a construction similar to that of test strip 30 is shown in FIG. 4. The sensor includes a working electrode 112 disposed on substrate 124. Electrodes 118, 120, and 122 are disposed on second substrate 128. Spacer layer 126 (an adhesive) separates working electrode 112 from electrodes 118, 120, and 122. 118 and 122 are trigger electrodes and 120 is a silver/silver chloride combined counter/reference electrode. Substrates 128, 124, in combination with spacer 126 define the sample chamber 114. Sample chamber 114 includes two entrances on side edges of the sensor, the entrance are marked by marking 114a and 114b. 110 depicts a sample as it is filled into the sample chamber 114. Sample chamber 114 includes the working electrode 112. The trigger electrode closest to the side where the sample has been applied indicates when the sample has started filling the sample chamber and the trigger electrode at the opposite side of the sample chamber indicates when the sample chamber has been filled by the sample.

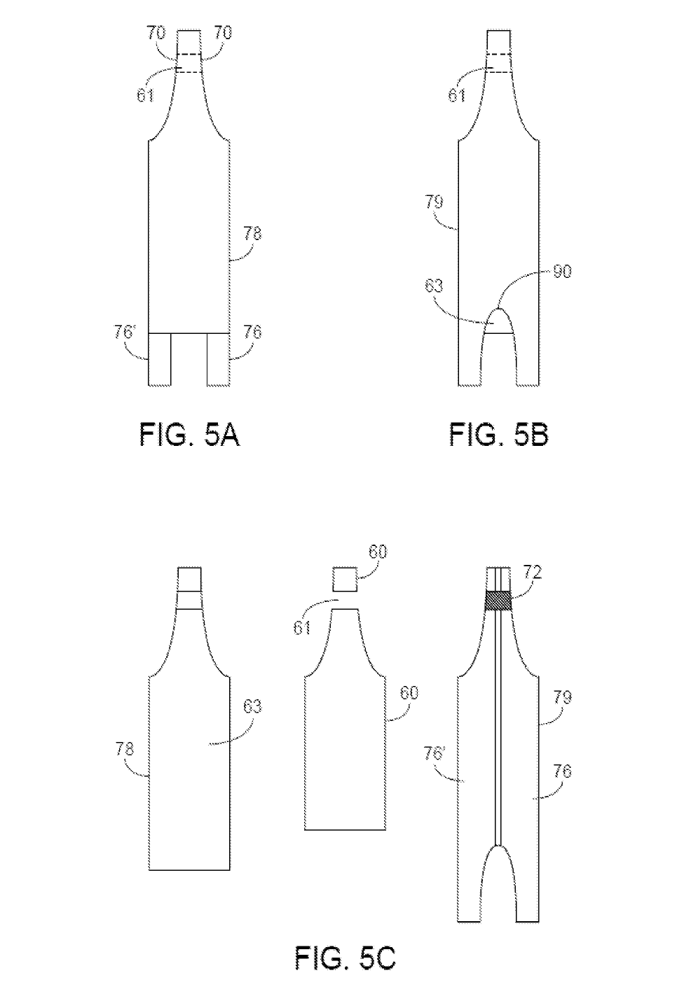

A test strip with electrodes in a facing configuration but with only three electrodes may also be used. The test strip is shown FIGS. 5A-5C. The sensor includes a first substrate 79 with two electrodes 76 and 76′ disposed on the substrate; a second substrate 78 with another electrode 63 disposed on it. A spacer layer 60 separates the electrodes 76 and 76′ from electrode 63. At the proximal end of the sensor, a sample chamber 61, defined by the two substrates and spacer 60, is located. The sample chamber includes two entrances (70) at opposite side edges of the sensor. At the distal end of the sensor, the substrate 79 includes a notch 90 that exposes the distal end of electrode 63, allowing it to connect to measuring device, e.g., a meter. Substrate 78 is shorter than substrate 79, exposing distal ends of electrodes 76 and 76′ for connection to a meter. A reagent system 72 is included in the sample chamber.

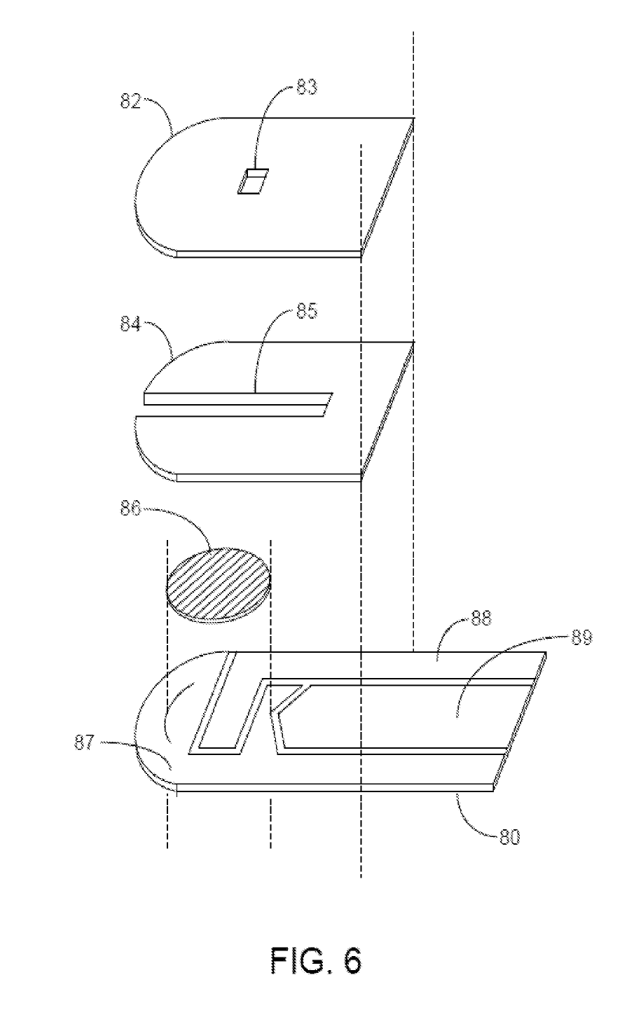

A test strip with electrodes in a coplanar configuration is also provided. An exploded view of the test strip is shown FIG. 6. Three electrodes 87, 88, and 89 are present on a lower substrate 80. Electrode 89 is the trigger electrode, electrode 87 is reference/counter electrode, and electrode 88 is working electrode. A reagent system 86 is disposed on electrodes 87 and 88. A spacer layer 84 with a cut out 85 provides a path for the sample. The upper substrate 82 includes an opening 83 to vent air as a sample fills the sample chamber defined by the upper and lower substrates and spacer layer.

It is noted that the term “substrate(s)” when used in context of the layers of the test strips refers to the support layers of the test strip and not to the substrate of an enzyme.

In certain embodiments, the height of the sample chamber may be about 100 μm, 50 μm, or 25 μm and the working electrode may be disposed in a facing configuration to the counter electrode (or counter/reference electrode). In certain cases, the working electrode may cover almost an entire side of the sample chamber. For example, the sample chamber may be defined by a first layer, a spacer layer, and a second layer, where the surface of the first layer present in the sample chamber may be entirely or substantially entirely covered with the working electrode.

As noted above, the test strip may include a first and a second layer and a first and a second electrode. In certain cases, one or both of the first, second, spacer layers and the electrodes may be substantially transparent or substantially opaque. As used herein, the phrase “substantially transparent” used in the context of layers (support layers of the test strip-first, second, and spacer layers), or electrodes refers to layers or electrodes which allow visualization of filling of sample chamber with a sample. In certain embodiments, the sensor strip may have a substantially transparent sample chamber such that the filling of the sample chamber with blood may be visualized. For example, the layers and the electrodes may allow for about 100%, or about 90%, or about 80%, or about 70%, or about 60%, or about 50%, or about 40% of light to pass through it.

The test strip includes a sample chamber at a proximal end of the strip, the sample chamber is defined by the first and second substrates and the spacer layer and includes a first and a second electrode. The distal end of the test strip is configured for insertion of the test strip into a measurement device. In certain embodiments, the first and second electrodes comprise contact tabs for connection to the measurement device. The contact tabs are located at or near the distal end of the test strip.

Redox Mediator

The sample chamber may include a redox mediator as discussed above. In one embodiment, the redox mediator is disposed on the working electrode. In certain cases, the mediator may be immobilized on the working electrode. Materials and methods for immobilizing a redox mediator on an electrode are provided in U.S. Pat. No. 6,592,745, the disclosure of which is incorporated by reference herein. In an alternative embodiment, the redox mediator is disposed adjacent to the working electrode.

Almost any organic or organometallic redox species can be used as a redox mediator. In general, suitable redox mediators are rapidly reducible and oxidizable molecules having redox potentials a few hundred millivolts above or below that of the standard calomel electrode (SCE), and typically not more reducing than about −200 mV and not more oxidizing than about +400 mV versus SCE. Examples of organic redox species are quinones and quinhydrones and species that in their oxidized state have quinoid structures, such as Nile blue and indophenol.

In certain cases, mediators suitable for use in the sensors have structures which prevent or substantially reduce the diffusional loss of redox species during the period of time that the sample is being analyzed. Suitable redox mediators include a redox species bound to a polymer which can in turn be immobilized on the working electrode. Useful redox mediators and methods for producing them are described in U.S. Pat. Nos. 5,262,035; 5,264,104; 5,320,725; 5,356,786; 6,592,745; and 7,501,053, the disclosure of each of which is incorporated by reference herein. Any organic or organometallic redox species can be bound to a polymer and used as a redox mediator. In certain cases, the redox species is a transition metal compound or complex. The transition metal compounds or complexes may be osmium, ruthenium, iron, and cobalt compounds or complexes. In certain cases, the redox mediator may be an osmium compounds and complex.

One type of non-releasable polymeric redox mediator contains a redox species covalently bound in a polymeric composition. An example of this type of mediator is poly(vinylferrocene).

Alternatively, a suitable non-releasable redox mediator contains an ionically-bound redox species. Typically, these mediators include a charged polymer coupled to an oppositely charged redox species. Examples of this type of mediator include a negatively charged polymer such as Nafion® (Dupont) coupled to a positively charged redox species such as an osmium or ruthenium polypyridyl cation. Another example of an ionically-bound mediator is a positively charged polymer such as quaternized poly(4-vinyl pyridine) or poly(l-vinyl imidazole) coupled to a negatively charged redox species such as ferricyanide or ferrocyanide.

In another embodiment, the suitable non-releasable redox mediators include a redox species coordinatively bound to the polymer. For example, the mediator may be formed by coordination of an osmium or cobalt 2, 2′-bipyridyl complex to poly(l-vinyl imidazole) or poly(4-vinyl pyridine).

The redox mediator may be osmium transition metal complexed with one or more ligands having a nitrogen-containing heterocycle such as 2,2′-bipyridine, 1,10-phenanthroline or derivatives thereof. Furthermore, the redox mediator may also have one or more polymeric ligands having at least one nitrogen-containing heterocycle, such as pyridine, imidazole, or derivatives thereof. These mediators exchange electrons rapidly between each other and the electrodes so that the complex may be rapidly oxidized and reduced.

In particular, it has been determined that osmium cations complexed with two ligands containing 2,2′-bipyridine, 1,10-phenanthroline, or derivatives thereof, the two ligands not necessarily being the same, and further complexed with a polymer having pyridine or imidazole functional groups form particularly useful redox mediators in the small volume sensors. Derivatives of 2,2′-bipyridine for complexation with the osmium cation may be 4,4′-dimethyl-2,2′-bipyridine and mono-, di-, and polyalkoxy-2,2′-bipyridines, such as 4,4′-dimethoxy-2,2′-bipyridine, where the carbon to oxygen ratio of the alkoxy groups is sufficient to retain solubility of the transition metal complex in water. Preferred derivatives of 1,10-phenanthroline for complexation with the osmium cation are 4,7-dimethyl-1,10-phenanthroline and mono-,di-, and polyalkoxy-1,10-phenanthrolines, such as 4,7-dimethoxy-1,10-phenanthroline, where the carbon to oxygen ratio of the alkoxy groups is sufficient to retain solubility of the transition metal complex in water. Exemplary polymers for complexation with the osmium cation include poly(l-vinyl imidazole), e.g., PVI, and poly(4-vinyl pyridine), e.g., PVP, either alone or with a copolymer. Most preferred are redox mediators with osmium complexed with poly(l-vinyl imidazole) alone or with a copolymer.

Suitable redox mediators have a redox potential between about −150 mV to about +400 mV versus the standard calomel electrode (SCE). For example, the potential of the redox mediator can be between about −100 mV and +100 mV, e.g., between about −50 mV and +50 mV. In one embodiment, suitable redox mediators have osmium redox centers and a redox potential more negative than +100 mV versus SCE, e.g., the redox potential is more negative than +50 mV versus SCE, e.g., is near −50 mV versus SCE.

In one embodiment, the redox mediators of the disclosed analyte sensors are air-oxidizable. This means that the redox mediator is oxidized by air, e.g., so that at least 90% of the mediator is in an oxidized state prior to introduction of sample into the sensor. Air-oxidizable redox mediators include osmium cations complexed with two mono-, di-, or polyalkoxy-2,2′-bipyridine or mono-, di-, or polyalkoxy-1,10-phenanthroline ligands, the two ligands not necessarily being the same, and further complexed with polymers having pyridine and imidazole functional groups. In particular, Os[4,4′-dimethoxy-2,2′-bipyridine]₂Cl^{+/+2} complexed with poly(4-vinyl pyridine) or poly(l-vinyl imidazole) attains approximately 90% or more oxidation in air.

In one specific embodiment, the redox mediator is 1,10 Phenanthrolene-5,6-dione (PQ).

To prevent electrochemical reactions from occurring on portions of the working electrode not coated by the mediator, a dielectric may be deposited on the electrode surrounding the region with the bound redox mediator. Suitable dielectric materials include waxes and non-conducting organic polymers such as polyethylene. Dielectric may also cover a portion of the redox mediator on the electrode. The covered portion of the mediator will not contact the sample, and, therefore, will not be a part of the electrode's working surface.

Although it can be advantageous to minimize the amount of redox mediator used, the range for the acceptable amount of redox mediator typically has a lower limit. The minimum amount of redox mediator that may be used is the concentration of redox mediator that is necessary to accomplish the assay within a desirable measurement time period, for example, no more than about 5 minutes, or no more than about 1 minute, or no more than about 30 seconds, or no more than about 10 seconds, or no more than about 5 seconds, or no more than about 3 seconds, or no more than about 1 second or less.

Fill Assist

The test strips can be configured for top-filling, tip-filling, corner-filling, and/or side-filling. In some embodiments, the test strips include one or more optional fill assist structures, e.g., one or more notches, cut-outs, indentations, and/or protrusions, which facilitate the collection of the fluid sample.

Exemplary test strips may include a sample chamber having an inlet with a projection extending from an edge of the sensor for facilitating flow of sample into the sample chamber. In certain cases, the sample chamber may extend from one side to another side of the test strip and may have two inlets, one at each side, with a projection extending from two edges of the sensor.

In certain cases, the test strips can be configured such that the proximal end of the test strip is narrower than the distal end of the test strip. In one such embodiment, the test strips includes a tapered tip at the proximal end of the test strips, e.g., the end of the test strips that is opposite from the end that engages with a meter. The sample chamber may extend from a side edge of the sensor to another side edge and the two inlets to the sample chamber may each include a protrusion which may be formed by an extension from one or both the first and second support layers of the test strip.

Exemplary fill assist structures are disclosed in U.S. Pat. No. 7,802,467, which is is incorporated by reference herein. Additional fill assist structures are described in U.S. Patent Publication No. 2008/0267823, the disclosure of which is incorporated by reference herein; and U.S. patent application Ser. No. 11/461,725, filed Aug. 1, 2006, the disclosure of which is incorporated by reference herein.

### Methods of Using Electrochemical Test Strips

The test strips described herein find use in methods for detecting enzyme activity and/or determining the amount of an enzyme in a sample, such as, a fluid sample from a subject. Generally, these methods include inserting a test strip into a measuring device such as a meter compatible with the test strip; contacting a fluid sample, e.g., a blood sample, with the test strip; and detecting a signal indicative of presence of the enzyme in the sample.

In certain cases, the method may include determining the amount of the enzyme using the generated signal. In general, the magnitude of the signal is proportional to the activity of the enzyme.

In certain embodiments, the method may include prior to or after the contacting a first test strip with a sample, contacting a second test strip with a control solution (e.g., a positive control solution) that includes a known amount of the enzyme. In certain cases, the second test strip is from the same lot/batch as the test strip used to assay enzyme activity in a body fluid sample.

In certain embodiments, the method may include prior to or after the contacting the sample, contacting a third test strip with a control solution (e.g., a negative control solution) that does not include the enzyme-thereby providing a background signal. In certain cases, the third test strip may be from the same lot/batch as the test strip used to assay enzyme activity in a body fluid sample.

In certain cases, the body fluid sample may not have a detectable enzyme activity. In such cases, the signal generated from the test strip upon application of the sample to the test strip may be insignificant, e.g., close to a background signal. In such a scenario, the sample may be tested using another test strip from the same lot/batch and/or from a different lot to confirm that the lack of a reliable signal is due to absence of the enzyme from the sample and not due to a defective test, such as, due a defect in the test strip. A positive control solution may facilitate with this confirmation and additionally confirm that the measuring device, such as, a meter is functioning accurately.

In certain embodiments, the measuring device may be a meter compatible with the test strip. For example, the meter may include contacts for connecting to the electrodes of the test strip. The test strip and the meter may be configured to provide a result of the assaying of enzyme activity within about 5 min after application of the sample to the sample chamber of the test strip, e.g., within about 4 min, 3 min, 2 min, 60 sec, 45 sec, 30 sec, 15 sec, or less.

In certain embodiments, the analyte test strip may turn the meter on or wake the meter up by depressing a switch in the meter, for example, a switch disposed in the sensor port of the meter. In other embodiments, the test strip may have a turn-on bar which wakes the meter up upon insertion of the test strip into a slot or port in the meter.

In certain embodiments, the meter may include a light source. For example, the meter may comprise a light source that lights up the port for the test strip. The light source may be present in the sensor port of the meter. The light source may illuminate a test strip inserted in the meter.

Analyte sensors and associated measuring devices that are known in the art may be configured to include the reagent system for detecting enzyme activity as described herein and used to perform the measurement of enzyme concentration as described above. Suitable analyte meters and other devices that may be configured as described above include, for example, those described in U.S. Pat. No. 7,041,468; U.S. Pat. No. 5,356,786; U.S. Pat. No. 6,175,752; U.S. Pat. No. 6,560,471; U.S. Pat. No. 5,262,035; U.S. Pat. No. 6,881,551; U.S. Pat. No. 6,121,009; U.S. Pat. No. 7,167,818; U.S. Pat. No. 6,270,455; U.S. Pat. No. 6,161,095; U.S. Pat. No. 5,918,603; U.S. Pat. No. 6,144,837; U.S. Pat. No. 5,601,435; U.S. Pat. No. 5,822,715; U.S. Pat. No. 5,899,855; U.S. Pat. No. 6,071,391; U.S. Pat. No. 6,120,676; U.S. Pat. No. 6,143,164; U.S. Pat. No. 6,299,757; U.S. Pat. No. 6,338,790; U.S. Pat. No. 6,377,894; U.S. Pat. No. 6,600,997; U.S. Pat. No. 6,773,671; U.S. Pat. No. 6,514,460; U.S. Pat. No. 6,592,745; U.S. Pat. No. 5,628,890; U.S. Pat. No. 5,820,551; U.S. Pat. No. 6,736,957; U.S. Pat. No. 4,545,382; U.S. Pat. No. 4,711,245; U.S. Pat. No. 5,509,410; U.S. Pat. No. 6,540,891; U.S. Pat. No. 6,730,200; U.S. Pat. No. 6,764,581; U.S. Pat. No. 6,299,757; U.S. Pat. No. 6,461,496; U.S. Pat. No. 6,503,381; U.S. Pat. No. 6,591,125; U.S. Pat. No. 6,616,819; U.S. Pat. No. 6,618,934; U.S. Pat. No. 6,676,816; U.S. Pat. No. 6,749,740; U.S. Pat. No. 6,893,545; U.S. Pat. No. 6,942,518; U.S. Pat. No. 6,514,718; U.S. Pat. No. 5,264,014; U.S. Pat. No. 5,262,305; U.S. Pat. No. 5,320,715; U.S. Pat. No. 5,593,852; U.S. Pat. No. 6,746,582; U.S. Pat. No. 6,284,478; U.S. Pat. No. 7,299,082; U.S. patent application Ser. No. 11/461,725, filed Aug. 1, 2006, entitled “Analyte Sensors and Methods”; U.S. Patent Application Publication No. US2004/0186365; U.S. Patent Application Publication No. 2007/0095661; U.S. Patent Application Publication No. 2006/0091006; U.S. Patent Application Publication No. 2006/0025662; U.S. Patent Application Publication No. 2008/0267823; U.S. Patent Application Publication No. 2007/0108048, the disclosures of each which are incorporated by reference herein.

In one embodiment, the concentration of the enzyme may be determined by amperometry, coulometry, potentiometry, and/or voltametry, including square wave voltametry, using the test strips and meters as described herein.

Prior to providing the sample to the sensor, or even after providing the sample to the sensor, there may be no need for the user to input a calibration code or other information regarding the operation and/or interaction of the sensor with the meter or other equipment. The sensor may be configured so that the results received from the analysis are clinically accurate, without the user having to adjust the sensor or the meter. For example, the sensor may be physically configured to provide accurate results that are repeatable by a batch of sensors.

After receipt of the sample in the sensor, the enzyme in the sample, e.g., electrooxidizes or electroreduces a substrate at the working electrode, where the level of current obtained is proportional to enzyme activity. The sensor may be operated with or without applying a potential to the working electrode. In one embodiment, the electrochemical reaction occurs spontaneously and a potential need not be applied between the working electrode and the counter electrode. In another embodiment, a potential is applied between the working electrode and the counter electrode.

In certain cases, the method may also include measuring the ambient temperature. In certain cases, the ambient temperature may be outside of the specified temperature limit. If the ambient temperature is outside the specified temperature limit, an error is reported. In certain cases, the ambient temperature may be measured by a measuring device into which the test strip is inserted by means of a temperature measurement instrument, such as, a thermometer, a thermistor, a pyrometer, a thermocouple, and the like. The temperature may be measured before applying a sample to the test strip. In certain cases, the temperature may be measured after applying a sample to the test strip.

As mentioned above, the meter may be a coulometer, a potentiometer or an amperometer. A meter may be available at generally the same locations as the test strips, and sometimes may be packaged together with the test strips, e.g., as a kit.

In certain cases, the meter or another measurement device connected to the test strips may be used to perform the methods described herein. In certain embodiments, the meter or measurement device, such as, hand-held reader, e.g., a reader module connectable to a personal device, such as, a smart phone, may include programming to calculate the enzyme activity. Examples of suitable electronics connectable to the meter include a data processing terminal, such as a personal computer (PC), a portable computer such as a laptop or a handheld device (e.g., personal digital assistants (PDAs)), and the like. The electronics are configured for data communication with the receiver via a wired or a wireless connection. The various devices connected to the meter may wirelessly communicate with a server device, e.g., using a common standard such as 802.11 or Bluetooth RF protocol, or an IrDA infrared protocol. The server device could be another portable device, such as a Personal Digital Assistant (PDA) or notebook computer, or a larger device such as a desktop computer, appliance, etc. In some embodiments, the server device has a display, such as a liquid crystal display (LCD), as well as an input device, such as buttons, a keyboard, mouse or touch-screen. The server device may also communicate with another device, such as for sending data from the meter and/or the service device to a data storage or computer. Examples of such communications include a PDA synching data with a personal computer (PC), a mobile phone communicating over a cellular network with a computer at the other end, or a household appliance communicating with a computer system at a physician's office.

The meter or another measuring device may include a display that may display the results of the assay-example, the measured activity if the enzyme or amount of the enzyme e.g., in enzyme Unit such as, U/ml or in katal. One katal is the amount of enzyme that converts 1 mole of substrate per second.

Prior to contacting of the test strip with a body fluid sample, the sample may be obtained from a subject. For example, a blood sample may be obtained from a finger of the subject. In certain cases, the sample may be blood obtained from a region of the subject having a lower nerve end density as compared to a fingertip, such as, palm, forearm, thigh, or abdomen.

The test strips of the present disclosure assist in testing a sample fluid from a patient at point-of-care, such as, a doctor's office, or a nurse's or nurse assistant's station. The test strips of the present disclosure provide a rapid assay of enzyme activity which aids the care provider in determining whether the enzyme of interest is present or absent in a patient. In certain embodiments, the test strips of the present disclosure provide a rapid measurement of the amount of the enzyme, if present, based on the measured enzyme activity.

The test strips of the present disclosure thus may facilitate treatment decisions, diagnosis, and/or prognosis of a patient.

In certain case, the test strip may be a test strip for assaying activity of G6PD in a sample from a subject. G6PD is part of the oxidative pentose pathway, wherein it functions to minimize oxidative attacks of free radicals upon cells by providing reducing equivalents. For example, G6PD converts glucose-6-phosphate to 6-phosphoglutonate, thereby, releasing a proton that reduces nicotinamide adenine dinucleotide phosphate (NADP) NADPH, a reduced form of NADP. The NADPH initiates a series of downstream reactions that ultimately reduce the free radical oxidizing agents and render many of them ineffective.

G6PD is present in most human cells, but it is in higher concentration in red blood cells which, in one of their primary function, act as oxygen transporter and are therefore particularly susceptible to oxidative attack. This enzyme helps protect red blood cells from oxidative damage and premature destruction. The efficiency of the G6PD system is remarkably high as reflected by the fact that, during normal activity, less than 1% of its capacity is utilized in combating and preventing undesirable oxidative reactions. However, when strong oxidizing agents, such as members of the quinine class of anti-malarial drugs must be introduced to human body, the need for rapid production of reducing agent is greatly increased.

Several mutations of the gene which encodes for G6PD are known. These mutations decrease the activity of the enzyme but not the expression level of the enzyme. In these individuals, administration of strong oxidizing agents such as members of the class of quinine-type anti-malarial drug may cause severe clinical complications, such as hemolytic anemia, because the low activity of their G6PD does not enable the production of sufficient reducing agents to prevent rapid unwanted oxidative damage on their red blood cells. In areas where malarial infections are common and at times even epidemic, a need therefore exists for a rapid efficient test that will readily distinguish persons having G6PD of low activity from persons whose G6PD activity is normal and will enable medical personnel to ensure that (1) the quinine anti-malarial drugs are prescribed only for individuals with at least a normal G6PD activity and (2) persons with lower than normal G6PD activity are medicated with an alternative type of anti-malarial drugs.

A test strip for assaying activity of lactate dehydrogenase may be used to assess level of lactate dehydrogenase present in a blood sample of a patient. The rapid point of care test may facilitate diagnosis of internal injury in a subject. A positive test—higher than normal level of LDH activity may indicate that the subject has internal injuries and should be referred to a hospital for treatment.

### Kits

Multiple test strips as disclosed herein may be packaged together and sold as a single unit; e.g., a package of about 25, about 50, or about 100 sensors, or any other suitable number. A kit may include one or more test strips, and additional components such as control solutions and/or lancing device and/or meter, etc.

A lancing device or other mechanism to obtain a sample of biological fluid, e.g., blood, from the patient or user may also be available at generally the same locations as the sensors and the meter, and sometimes may be packaged together with the sensor and/or meter, e.g., as a kit.

### Methods of Making Analyte Sensors

Analyte sensor or sensor strips discussed above, are sandwiched or layered constructions having first and second substrates spaced apart by a spacer layer and optionally including a mesh layer in the sample chamber defined by the first and second substrates and the spacer layer. Such a construction can be made by combining the various layers together in any suitable manner. An alternate method for making sensor strips as described herein is to mold the sensors.

In general, the method of manufacturing sensor strips involves positioning a working electrode and a reference and/or a counter electrode on the first or the second substrates, contacting at least a portion of the working electrode and/or reference and/or counter electrode with the reagent system as described herein.

Optionally, providing a mesh in the sample chamber defined by the first and second substrates and the spacer layer.

The following examples are put forth so as to provide those of ordinary skill in the art with a complete disclosure and description of how to make and use the embodiments of the invention, and are not intended to limit the scope of what the inventor regards as the invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Test Strip for Detecting Glucose-6-Phosphate Dehydrogenase

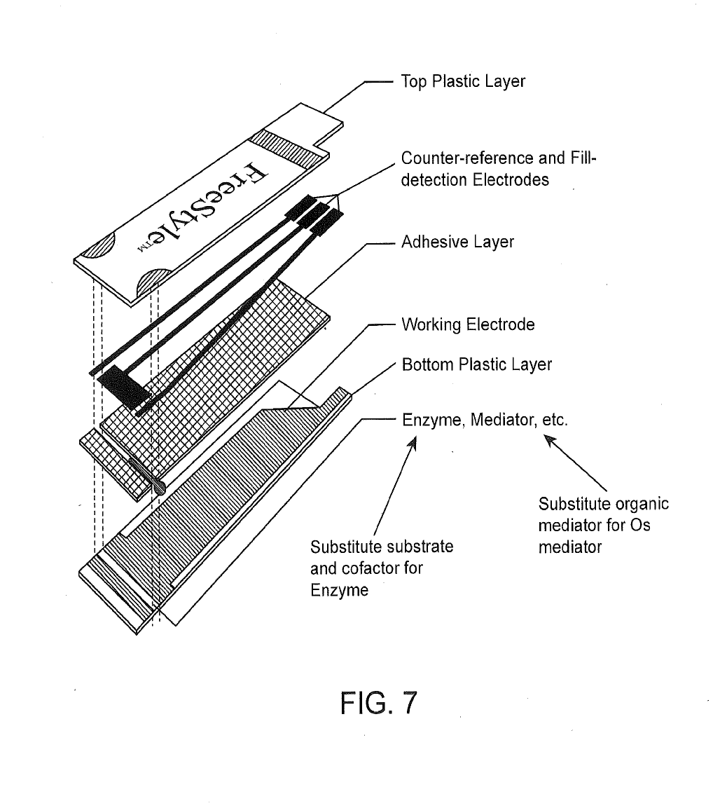

FreeStyle test strip was modified into a glucose-6-phosphate dehydrogenase (G6PDH) enzyme-detecting strip. In lieu of the glucose dehydrogenase enzyme, the substrate of G6PDH, glucose-6-phosphate, was added to the sample chamber. NADP, a cofactor of G6PDH, was also added to the strip. In addition, instead of the Os mediator (MAP), phenazine methosulfate (PMS), a mediator which is compatible with NAD-dependent enzymes, was added. These substitutions are illustrated in FIG. 7.

The formulation, per strip, was as follows:

Working electrode chemistry

| | Amount |
|---|---|
| Compound | (μg/strip) |
| Phenazine methosulfate | 0.25 |
| NADP+ | 1.5 |
| Glucose-6-phosphate | 5 |
| FS30 | 3 |
| Boric Acid | 1.5 |

Counter electrode chemistry

| | Amount |
|---|---|
| Compound | (μg/strip) |
| Hepes (Buffer) | 6 |
| FS30 | 3 |

These test strips were tested in standard FreeStyle Freedom meters (with an applied potential of +100 mV), by adding aliquots of G6PDH-containing buffer solutions to the strips, and monitoring the resulting currents for 60 s. Each concentration of G6PDH, from 0 to 5 U/mL (in 100 mM PBS, 0.1% TRITON X-100), was tested on duplicate strips.

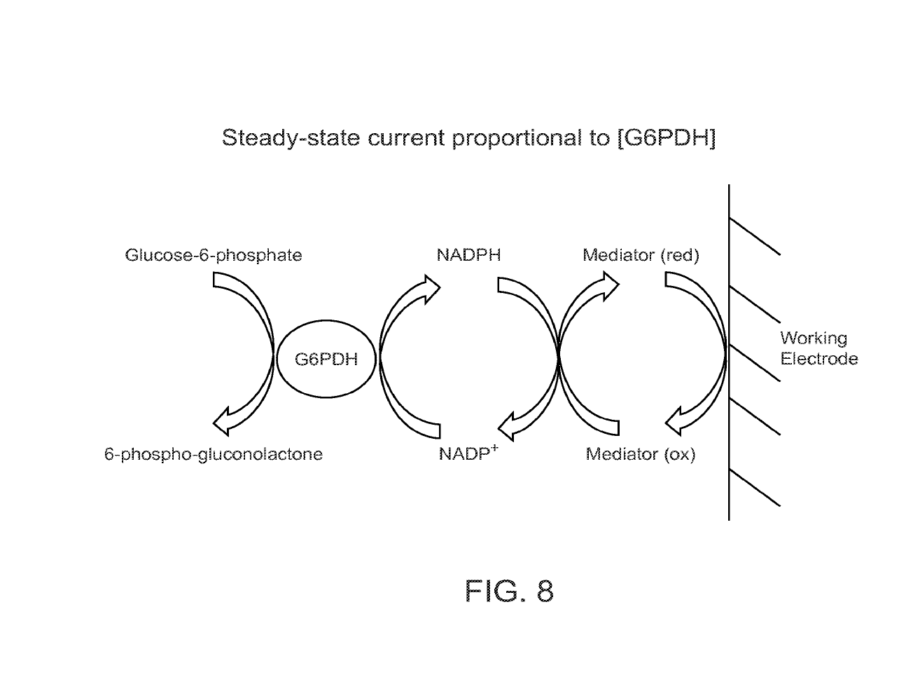

The internal strip process, in the case of a G6PDH assay, is illustrated in FIG. 8. As shown in FIG. 8, Glucose-6-phosphate derived electrons are passed sequentially to G6PDH, to NADP+, to phenazine methosulfate and to the electrode. Electrons re-emerge at the counter-reference electrode (not shown) to convert AgCl to Ag and dissolved Cl⁻, completing the electrochemical circuit. Since the substrate, cofactor, and mediator are in excess, the reaction is enzyme limited, and results in a steady state current proportional to the enzyme activity.

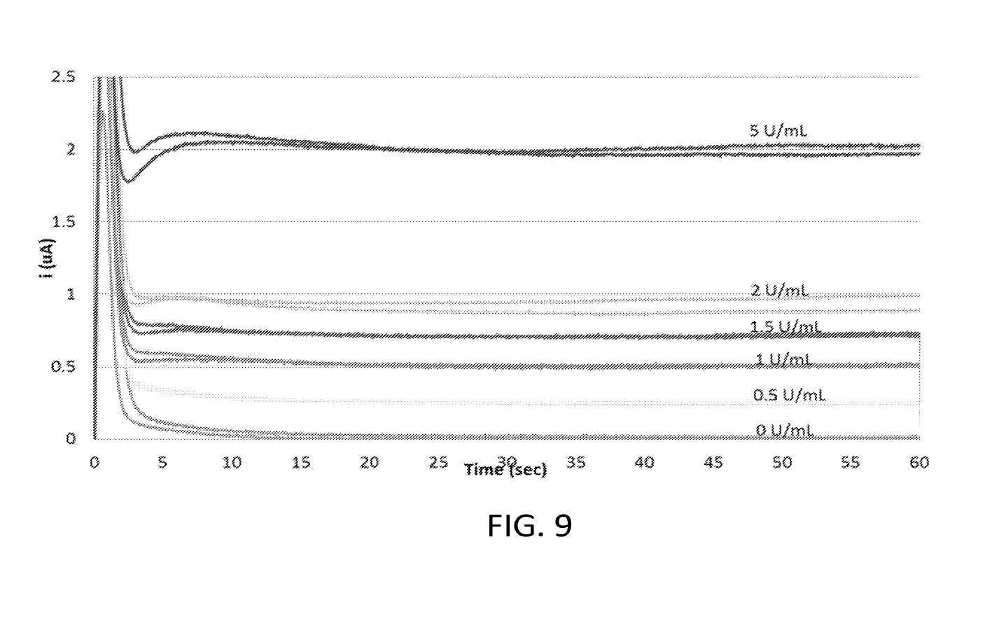

The current profile measured by the meter over a 60 s time period after application of the solutions to the modified test strips is provided in FIG. 8. As shown in FIG. 9, after an initial current spike, the current equilibrates to a steady state value, which is reproducible at a given G6PDH concentration. The intercept (0 U/mL) is also desirably low. Equilibration time for these strips was about 20 s.

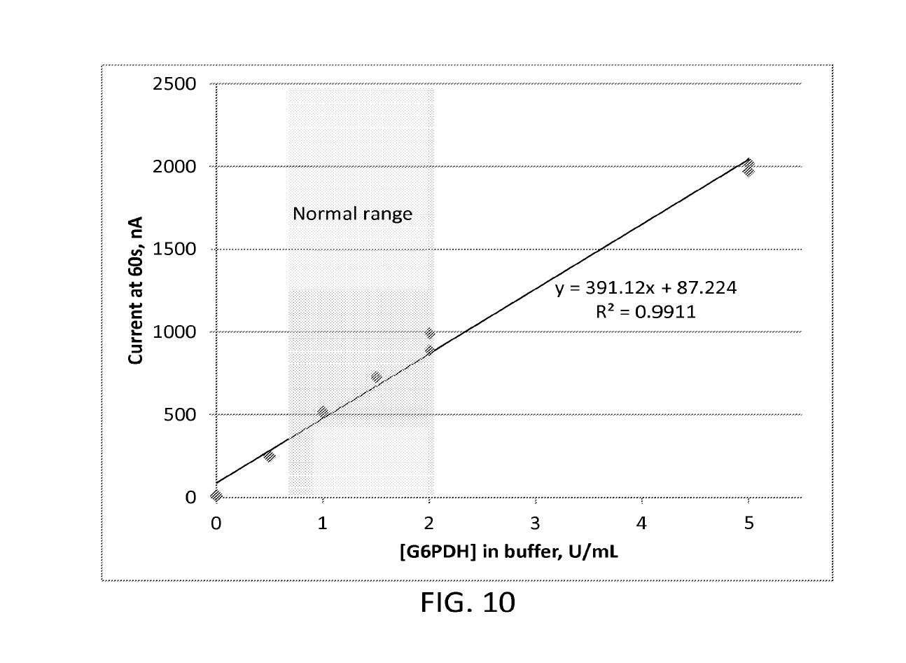

The steady-state current value was plotted against G6PDH concentration. The resulting graph is shown in FIG. 10. Notably, the curve is linear, encompasses the normal range present in human blood, and has sufficient sensitivity (slope) to detect reduced G6PDH.

### Example 2

### Test Strip with Diaphorase and Osmium-Based Mediator for Detecting G6PDH

Addition of Diaphorase and Os-based mediators. In general, Os-based mediators cannot efficiently oxidize NAD+, and this is the reason that the original formulation, described above, substituted PMS for an Os-based mediator. However, PMS (and many organic mediators) suffer from the disadvantage that they react fairly rapidly with oxygen, which is especially problematic in oxygen-rich blood. Therefore it would be desirable to retain an Os-based mediator (or another mediator inert to oxygen), in place of the organic mediator.

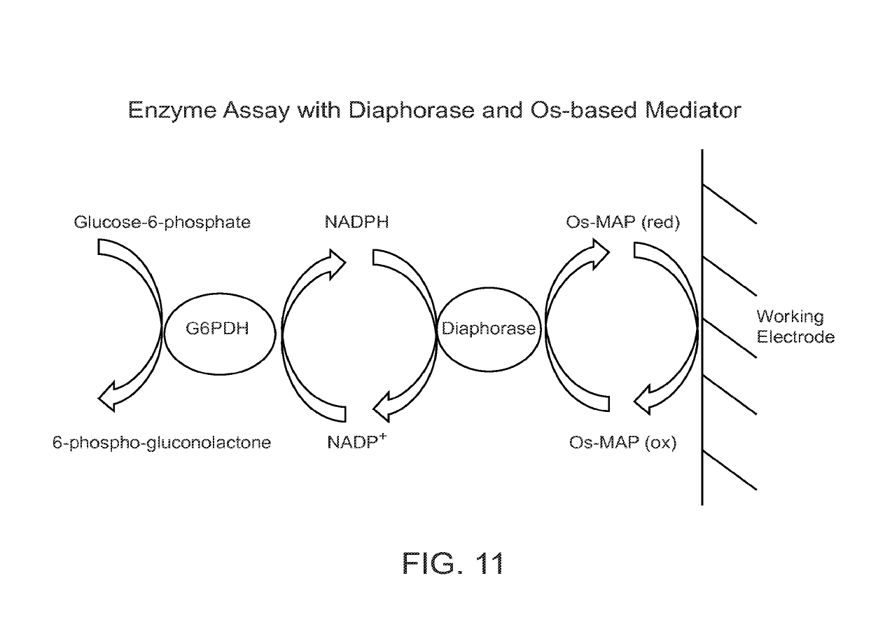

This was be accomplished by adding an enzyme called diaphorase, which mediates between an Os-based mediator and NAD+. This eliminates the need for an organic mediator, as shown in the FIG. 11.

The modified test strip included the following formulation:

Working Electrode Chemistry

| | Amount |
|---|---|
| Compound | (U/strip) |
| Diaphorase | 0.0058 |
| | Amount |
| Compound | (micrograms/strip) |
| NADP+ | 0.38 |
| Glucose-6-phosphate | 0.75 |
| MAP | 0.38 |
| FS30 | 3 |
| Pipes (Buffer) | 4.25 |

Counter Electrode Chemistry

| | Amount |
|---|---|
| Compound | (micrograms/strip) |
| Hepes (Buffer) | 6 |
| FS30 | 3 |

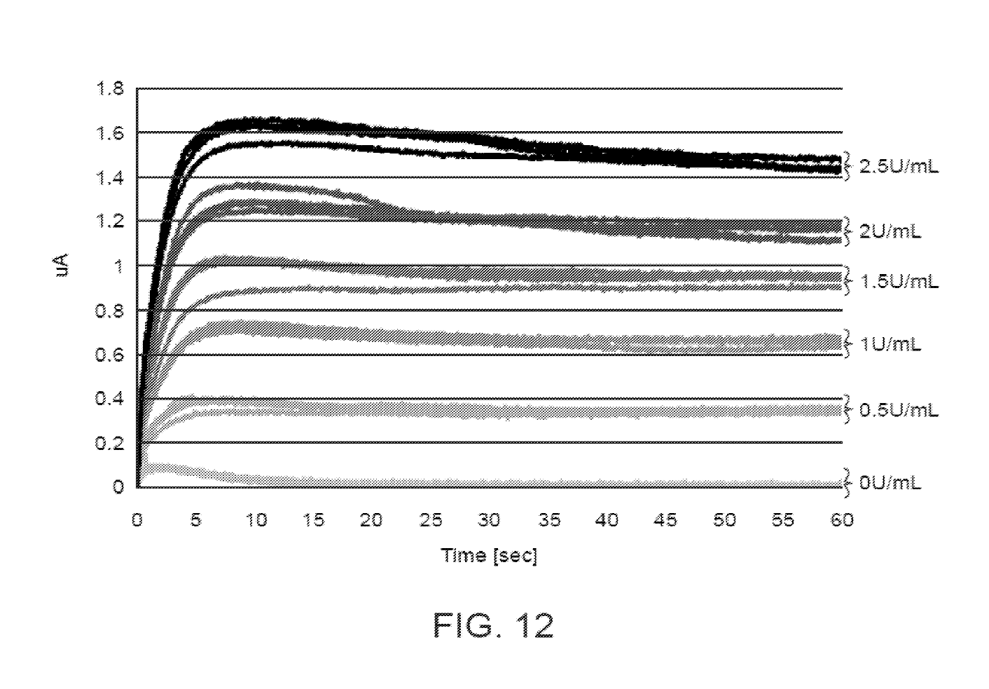

These test strips were tested in standard FreeStyle Freedom meters (with an applied potential of +100 mV), by adding aliquots of G6PDH-containing buffer (PBS) solutions to the strips, and monitoring the resulting currents for 60 s. Each concentration of G6PDH, from 0 to 5 U/mL, was tested on duplicate strips. The current profile generated over the 60 s time period is shown in FIG. 12.

The linearity of the curves shown in FIG. 12 demonstrates that diaphorase and Os-MAP have been successfully substituted for PMS in the assay.

### Example 3

### Test Strip for Detecting G6PDH in Whole Blood

Testing in Whole Blood.

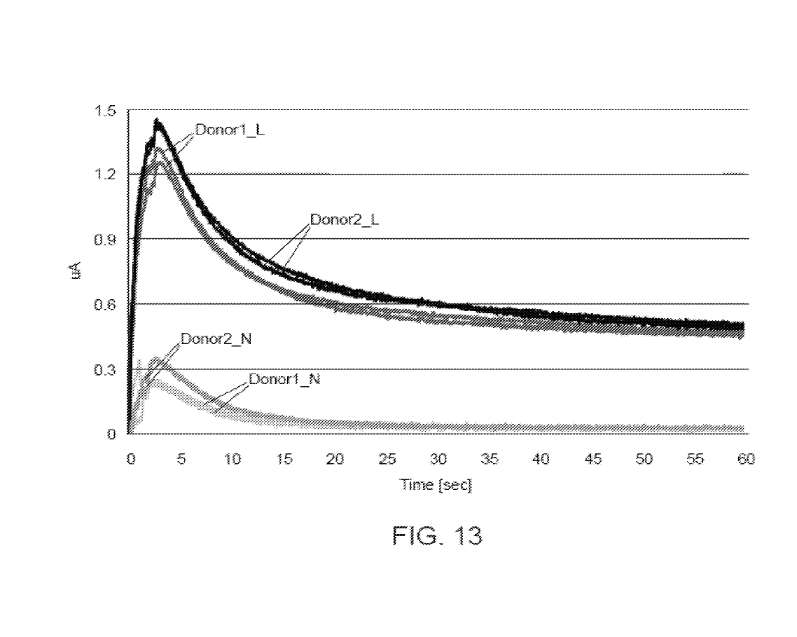

G6PDH is present in erythrocytes. In certain cases, the erythrocytes are lysed to release the enzyme. Erythrocytes can be lysed prior to loading the blood sample into the test strip. This is illustrated by observing the output of G6PDH enzyme assay using both whole and lysed blood, from two different blood donors (Donor 1 and Donor 2). The current profile measured over a period of 60 s is shown in FIG. 13.

The samples from lysed blood (Donor 1_L and Donor 2_L) show substantial steady state currents of about 0.5 microamps, while the whole blood samples (Donor 1_N and Donor 2_N) show negligible current—near the baseline. This is expected, since the majority of G6PDH resides in the red blood cell, and is not accessible in whole blood.

Perform the lysing step inside the strip itself would greatly simplify assaying G6PDH enzyme activity. In order to perform the lysing step inside the test strip, a lysing agent, saponin, was placed in the strip, as described in the following formulation:

Working Electrode Chemistry

| | Amount |
|---|---|
| Compound | (U/strip) |
| Diaphorase | 0.0058 |
| | Amount |
| Compound | (μg/strip) |
| NADP+ | 0.38 |
| Glucose-6-phosphate | 0.75 |
| MAP | 0.38 |
| Saponin | 10 |
| FS30 | 3 |
| Pipes (Buffer) | 4.25 |

Counter Electrode Chemistry

| | Amount |
|---|---|
| Compound | (μg/strip) |
| Hepes (Buffer) | 6 |
| Triton X-100 | 10 |
| Saponin | 10 |

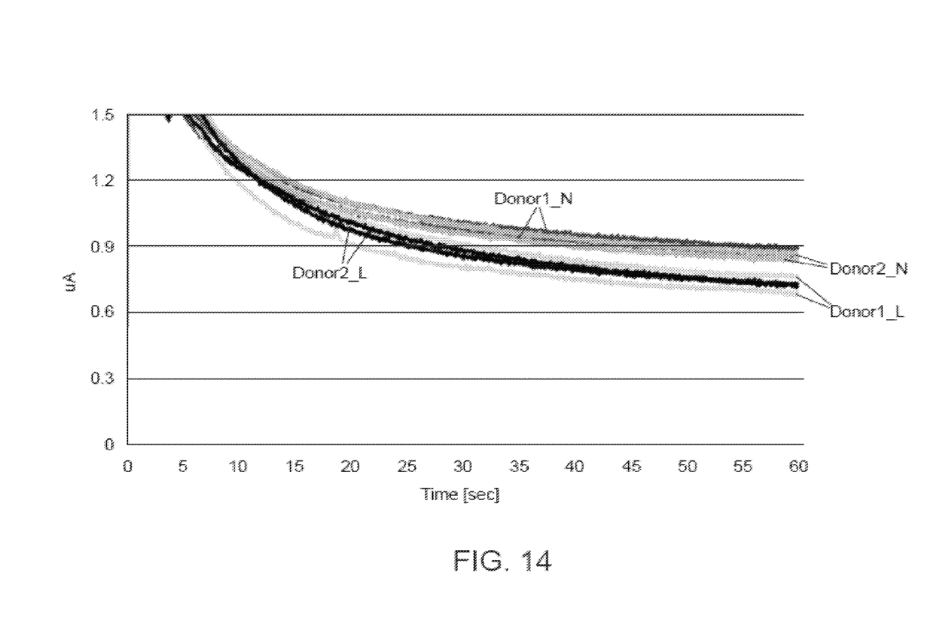

The results from test strips with this formulation, when tested with both lysed and whole blood, are shown in FIG. 14.

As seen in FIG. 14, both the lysed blood (Donor 1_L and Donor 2_L) and the whole blood (Donor 1_N and Donor 2_N) both now gave similar and substantial currents, indicating the G6PDH has been effectively released from the erythrocytes by the in-strip lysing agent.

Each of the various references, presentations, publications, provisional and/or non-provisional U.S. patent applications, U.S. patents, non-U.S. patent applications, and/or non-U.S. patents that have been identified herein, is incorporated herein by reference in its entirety.

Other embodiments and modifications within the scope of the present disclosure will be apparent to those skilled in the relevant art. Various modifications, processes, as well as numerous structures to which the embodiments of the invention may be applicable will be readily apparent to those of skill in the art to which the invention is directed upon review of the specification. Various aspects and features of the invention may have been explained or described in relation to understandings, beliefs, theories, and/or underlying assumptions, although it will be understood that the invention is not bound to any particular understanding, belief, theory, and/or underlying assumption. Although various aspects and features of the invention may have been described largely with respect to applications, or more specifically, medical applications, involving humans, it will be understood that such aspects and features also relate to any of a variety of applications involving any and all other animals. In addition, although embodiments of test strips and measuring devices such as meters may be described in separate embodiments, the different embodiments of the test strips and meter may be combined to provide a measurement system. Similarly, different aspects of different sensors and meters that may have been described in separate embodiments may be combined together. Finally, although the various aspects and features of the invention have been described with respect to various embodiments and specific examples herein, all of which may be made or carried out conventionally, it will be understood that the invention is entitled to protection within the full scope of the appended claims.

1. An electrochemical test strip for assaying activity of an enzyme, comprising:
a first layer, a second layer and a spacer layer interposed between the first and second layers;
a sample chamber defined by the first, second, and spacer layers, and positioned between the first and second layers;
the sample chamber comprising:
a substrate for the enzyme and a redox mediator disposed in the sample chamber; and
a first electrode and a second electrode.

2. The test strip of claim 1, wherein the substrate is glucose-6-phosphate and the enzyme is glucose-6-phosphate dehydrogenase.

3. The test strip of claim 1, wherein the sample chamber comprises an erythrocyte lysing agent.

4. The test strip of claim 1, wherein the substrate is glucose-6-phosphate, lactate, hemoglobin, pyruvate, creatinine, aspartate, alanine, urea, or cholesterol.

5. The test strip of claim 1, wherein the first layer or the second layer is transparent.

6. The test strip of claim 1, wherein the first and second electrodes are disposed on the first layer or the second layer.

7. The test strip of claim 1, wherein one of the electrodes is disposed on the first layer and the other electrode is disposed on the second layer.

8. The test strip of claim 1, wherein a third electrode is present in the sample chamber.

9. The test strip of claim 8, wherein the first, second, and third electrodes are working, counter, and reference electrodes, respectively.

10. The test strip of claim 1, wherein the first electrode is a working electrode and the second electrode is a counter electrode, wherein the electrodes independently comprise a material selected from the group consisting of: gold, carbon, platinum, tin oxide, ruthenium, palladium, silver, silver chloride, silver bromide, and combinations thereof.

11. The test strip of claim 10, wherein the counter electrode comprises Ag/AgCl.

12. The test strip of claim 1, wherein the first electrode is a working electrode and is disposed on the first layer and the second electrode is a counter electrode and is disposed on the second layer.

13. The test strip of claim 1, wherein the first electrode is a working electrode and the second electrode is a counter electrode and wherein both are disposed on the first layer or on the second layer.

14. The test strip of claim 1, wherein the substrate and redox mediator are disposed on the first electrode or the second electrode.

15. The test strip of claim 1, wherein the redox mediator is phenazine methosulfate.

16. The test strip of claim 15, wherein the redox mediator comprises a transition metal complex.

17. The test strip of claim 16, wherein the transition metal complex comprises a transition metal selected from the group consisting of osmium, ruthenium, iron and cobalt.

18. The test strip of claim 17, wherein the transition metal is osmium.

19. The test strip of claim 1, wherein the sample chamber is sized to contain a volume of no more than about 1 μL of sample fluid.

20. The test strip of claim 1, wherein the sample chamber is sized to contain a volume of no more than about 0.5 μL of sample fluid.

21. The test strip of claim 1, wherein the sample chamber comprises a cofactor for the enzyme.

22. The test strip of claim 1, wherein the sample chamber has a height of about 200 μm to about 25 μm.

23. The test strip of claim 1, wherein the sample chamber has a height of about 100 μm to about 40 μm.

24. The test strip of claim 1, wherein the sample chamber has a height of about 75 μm to about 50 μm.

25. A kit comprising:
the test strip of claim 1; and
a control solution comprising the enzyme.

26. A system for assaying activity of an enzyme, comprising:
the test strip of claim 1; and
a meter compatible with the test strip, the meter comprising electronics for measuring a signal generated by action of the enzyme on the substrate.

27. The system of claim 26, wherein the signal is selected from a group consisting of current, resistance, impedance, voltage, and capacitance.

28. The system of claim 26, wherein the meter comprises a display for displaying the results of the assaying the activity of the enzyme.

29. A method for assaying activity of an enzyme, comprising:
contacting a sample suspected of containing the enzyme with a first test strip, wherein the first test strip is the test strip of claim 1; and
measuring a signal generated by action of the enzyme on the substrate,
wherein the presence of the signal indicates that the enzyme is present in the sample; and
wherein magnitude of the signal is proportional to the concentration of the enzyme present in the sample.

30. The method of claim 29, wherein prior to or after the contacting the sample, the method comprises contacting a second test strip with a control solution.

31. The method of claim 29, wherein the method further comprises determining concentration of the enzyme by coulometry using the signal.

32. The method of claim 29, wherein the method further comprises determining concentration of the enzyme by amperometry using the signal.

33. The method of claim 29, wherein the sample is a body fluid of a subject.

34. The method of claim 33, wherein the sample is whole blood.

35. The method of claim 34, wherein the whole blood is obtained from the finger of the subject.

36. The method of claim 33, wherein the sample is blood obtained from a region of the subject having a lower nerve end density as compared to a fingertip.
